(19) United States
(12) Patent Application Publication
(10) Pub. No.: US 2026/0191674 A1
(43) Pub. Date: Jul. 9, 2026

(54) POSTURE CORRECTION DEVICE

(71) Applicant: PRANASH (TIANJIN) TECHNOLOGY DEVELOPMENT CO., LTD., Tianjin (CN)

(72) Inventor: Dongmei WEI, Tianjin (CN)

(21) Appl. No.: 19/551,200

(22) Filed: Feb. 26, 2026

(30) Foreign Application Priority Data
Oct. 11, 2025 (CN) 202522149732.3

(51) Int. Cl. A61F 5/02

(52) U.S. Cl. CPC A61F 5/026; A61F 5/028

(57) ABSTRACT

A posture correction device, including: a main orthopedic band, two abdominal bands having a hook and loop fastener loop surface area symmetrically and adjustably connected to the main orthopedic band. Two shoulder straps connected to the main orthopedic band. A back limiting portion fixedly disposed on a side of the main orthopedic band facing away from a body of the user. Two pulling straps having first ends connected to the two shoulder straps, and second ends pass through the back limiting portion and extends downward, and cross each other in an X-shape. Distal end lower surfaces of the two pulling straps are provided with a hook and loop fastener hook surface. A lumbar pressure band connected to the main orthopedic band, the lumbar pressure band including a support component and elastic connecting straps, the elastic connecting straps are connected to the support component and the two abdominal bands.

### CROSS-REFERENCE TO RELATED APPLICATIONS

This application claims foreign priority of Chinese Patent Application No. 202522149732.3, filed on October 11, 2025 in the China National Intellectual Property Administration, the disclosures of all of which are hereby incorporated by reference.

### TECHNICAL FIELD

The present disclosure relates to the technical field of ergonomic posture correction devices, in particular to a posture correction device.

### BACKGROUND

In recent years, posture correction products have received increasing attention. However, existing posture correction devices frequently encounter a conflict between functional integration and personalized adaptation in their design. Most conventional products adopt an integrated structure in which shoulder-back traction and waist-abdomen support functions are fixedly combined. While such designs aim to provide comprehensive correction, they suffer from significant limitations.

First, they are incapable of flexibly switching functions according to the user's real-time correction priorities (for example, when only shoulder relaxation is required or when lumbar support needs to be independently enhanced), resulting in poor adaptability.

Second, the pressure and shape of the supporting components are fixed, making it difficult to achieve personalized matching with respect to different users' lumbar physiological curvature or tolerance level, which readily leads to pressure points or insufficient support.

Third, damage or aging of any local portion may cause the entire product to fail, thereby reducing the product's economic efficiency and sustainability.

Particularly prominent is the situation where, when focused adjustment of the lumbar support scheme is required, the user often has no choice but to replace the entire posture correction device or is forced to accept a fixed pressure distribution that is not custom-tailored to their needs. This lack of a modular and customizable design philosophy severely restricts the product's capacity for precise corrective efficacy and long-term user experience.

### SUMMARY

In order to overcome the deficiencies of the prior art, the present disclosure provides a posture correction device characterized by structural innovation, scientifically sound support, and ease of personalized adjustment, which addresses the difficulty existing in conventional posture correction devices of effectively balancing systemic posture correction, fine stepless adjustment, and long-term wearing comfort. More specifically, the present disclosure seeks to solve, by means of a detachably connectable modular lumbar pressure band design, the problems of conventional integrated posture correction devices, namely their inability to flexibly adjust the lumbar support scheme according to individualized user needs, as well as their fixed and singular corrective functions and poor adaptability, thereby realizing coordinated, precise, and comfortable active correction of the shoulder, back, waist, and abdominal regions.

To realize the above objective, the present disclosure provides a posture correction device, including: a main orthopedic band having at least two main support rods embedded therein along a longitudinal direction of a spine, the at least two main support rods being parallel arranged to each other; two abdominal bands symmetrically and adjustably connected to lower portions of both sides of the main orthopedic band, the two abdominal bands being configured to encircle an abdomen of a user, each of the two abdominal bands having an outer surface provided with a hook and loop fastener loop surface area; two shoulder straps symmetrically and fixedly connected to upper portions of both sides of the main orthopedic band; a back limiting portion fixedly disposed on a side of the main orthopedic band facing away from a body of the user; two pulling straps, each of the two pulling straps having a first end connected to a back portion of a corresponding one of the two shoulder straps, and a second end of each of the two pulling straps sequentially slidably passes through the back limiting portion and extends downward, wherein the two pulling straps cross each other in an X-shape over the back limiting portion; and a distal end lower surface of each of the two pulling straps being provided with a hook and loop fastener hook surface; a lumbar pressure band detachably connected to the main orthopedic band, the lumbar pressure band comprising a support component and a plurality of elastic connecting straps, wherein a proximal end of each of the plurality of elastic connecting straps is connected to the support component, and a distal end thereof is detachably connected to a corresponding one of the two abdominal bands; wherein a restraining state of each of the two shoulder straps on a shoulder of the user is adjusted by changing an attachment position of the hook and loop fastener hook surface at the distal end of each of the two pulling straps relative to the hook and loop fastener loop surface area of a corresponding one of the two abdominal bands.

Furthermore, the support component is provided with a plurality of connection sections; the proximal end of each of the plurality of elastic connecting straps is connected to a respective one of the connection sections, and a number of the elastic connecting straps is greater than or equal to a number of the connection sections.

Furthermore, the number of the elastic connecting straps is an even number, and the elastic connecting straps are symmetrically arranged along a longitudinal centerline of the support component.

Furthermore, the number of the elastic connecting straps is at least four.

Furthermore, the number of the connection sections is at least three.

Furthermore, the main orthopedic band is provided with at least two auxiliary support mechanisms, symmetrically disposed on both sides of the main support rods.

Furthermore, the auxiliary support mechanisms are auxiliary support buckles, and each of the auxiliary support buckles removably accommodates an auxiliary supporting rod.

Furthermore, each of the auxiliary support rods comprises an arcuate segment adapted to a physiological curvature of a lumbar spine of the user.

Furthermore, each of the two abdominal bands is provided with a rib limiting strap, and after passing through the back limiting portion, each of the two pulling straps further passes through the rib limiting strap positioned on the same side.

Furthermore, the hook and loop fastener loop surface area of each of the two abdominal bands covers a major portion of its length, thereby facilitating continuous positional adjustment of the hook and loop fastener hook surface along a length direction of a corresponding one of the two abdominal bands.

Furthermore, a material of the rib limiting strap and the back limiting portion is microfiber leather.

Furthermore, the lumbar pressure band is detachably connected to the main orthopedic band by a hook and loop fastener assembly.

Furthermore, an outer surface of the main orthopedic band is provided with a hook and loop fastener loop surface, and a surface of the support component is provided with a hook and loop fastener hook surface.

Furthermore, an abdominal band support buckle is provided at each of the two abdominal bands close to the main orthopedic band.

Furthermore, an abdominal band support rod is detachably disposed in the abdominal band support buckle.

Furthermore, at least one shaping support rod is embedded at a junction between the two abdominal bands and the main orthopedic band.

Furthermore, a shape of the support component is X-shaped, Y-shaped, or inverted Y-shaped.

Furthermore, a material of the support component is leather.

Furthermore, the pulling strap is made of elastic webbing.

Furthermore, main bodies of the main orthopedic band, the two abdominal bands, and the two shoulder straps are made of flexible textile material.

The present disclosure provides the following advantageous effects.

Through the arrangement of the technical solution described above, during use: on one hand, two elastic pulling straps are threaded in an X-shaped crossing configuration through a back limiting portion, thereby optimally converting the rearward-downward pulling force exerted on the shoulders by the two shoulder straps into concentrated, symmetrical inward gathering forces acting on the medial borders of both scapulae, effectively guiding scapular adduction and depression. At the same time, by changing the attachment position of the hook and loop fastener hook surface at the distal end of each pulling straps, continuous and fine linear adjustment of both the magnitude and the point of application of the corrective force can be achieved, thereby overcoming the problems of coarse adjustment and inaccurate force lines inherent in conventional designs.

On the other hand, the lumbar pressure band is detachably connected to the main orthopedic band by means of hook and loop fasteners. The combination of a support component and a plurality of elastic connecting straps enable effective distribution of the pressure applied to the lumbar vertebrae, avoiding localized compression. The user can independently replace or adjust this module according to the depth of their own lumbar curvature and support requirements, thereby realizing an upgrade from “fixed integration” to “on-demand customization,” which significantly enhances the specificity and comfort of correction.

At the same time, longitudinal rigid stability is provided to the back by the main orthopedic band together with a main support rod; dynamic rearward-downward traction is provided to the shoulders by an elastic traction system; and adjustable anterior pressurization and stabilization are provided to the waist by the detachable pressure band. These three components interact and cooperate with one another to jointly form a mechanical framework capable of providing scientifically sound support in both the sagittal plane and the coronal plane, thereby achieving systematic correction of composite poor postures such as rounded shoulders, kyphosis, and abdominal/waist laxity.

Furthermore, the primary load-bearing components are fabricated from flexible textile materials, which ensure support performance while simultaneously providing overall wearing breathability and freedom of movement.

### BRIEF DESCRIPTION OF DRAWINGS

In order to more clearly illustrate the technical solutions in the embodiments of the present disclosure, a brief introduction is given below to the accompanying drawings required for describing the embodiments. The drawings described below are merely some embodiments of the present disclosure. For those of ordinary skill in the art, other drawings may also be obtained based on these drawings without any creative effort. In addition, the drawings are not drawn to a 1:1 scale, and the relative dimensions of the various elements in the drawings are shown merely by way of example and are not necessarily drawn to actual scale.

The present disclosure will be further described below in conjunction with the accompanying drawings and specific embodiments.

FIG. 1 is a front view of a posture correction device according to a first embodiment of the present disclosure, shown in a use state.

FIG. 2 is a rear view of the posture correction device according to the first embodiment of the present disclosure, show in the use state.

FIG. 3 is a side view of the posture correction device according to the first embodiment of the present disclosure in a use state.

FIG. 4 is a structural diagram of the posture correction device according to the first embodiment of the present disclosure, shown in a flat-laid state;

FIG. 5 is an exploded view of a portion of the posture correction device according to the first embodiment of the present disclosure, shown in the flat-laid state;

FIG. 6 is an enlarged view of a region circled A in FIG. 5;

FIG. 7 is a structural diagram of a posture correction device according to a second embodiment of the present disclosure, shown in a use state;

FIG. 8 is a structural diagram of the posture correction device according to the second embodiment of the present disclosure, shown in a flat-laid state;

FIG. 9 is a structural diagram of a posture correction device according to a third embodiment of the present disclosure, shown in a use state;

FIG. 10 is a structural diagram of the posture correction device according to the third embodiment of the present disclosure, shown in a flat-laid state.

### Description of the reference numeral

1 main orthopedic band; 2 auxiliary support buckle; 3 main support rod; 4 abdominal band; 41 abdominal band support buckle; 42 abdominal band support rod; 43 second opening; 5 shaping support rod; 6 rib limiting portion; 7 shoulder strap; 8 pulling strap; 9 back limiting portion; 10 lumbar pressure band; 1001 support component; 1001a connection section; 1002 elastic connecting strap; 11 auxiliary support rod; 12 first opening.

### DETAILED DESCRIPTION OF THE EMBODIMENTS

To make the above-mentioned objectives, features, and advantages of the present disclosure more apparent and readily understandable, the specific embodiments of the present disclosure are described in detail below in conjunction with the accompanying drawings. In the following description, numerous specific details are set forth in order to provide a thorough understanding of the present disclosure. However, the present disclosure can be implemented in many other ways different from those described herein, and a person skilled in the art may make similar improvements without departing from the spirit of the present disclosure. Therefore, the present disclosure is not limited to the specific embodiments disclosed below.

In the description of the present disclosure, it should be understood that when the following terms appear—“center,” “longitudinal,” “lateral,” “length,” “width,” “thickness,” “upper,” “lower,” “front,” “rear,” “left,” “right,” “vertical,” “horizontal,” “top,” “bottom,” “inner,” “outer,” “clockwise,” “counterclockwise,” “axial,” “radial,” “circumferential,” etc.—the orientation or positional relationships indicated by these terms are based on the orientation or positional relationships shown in the accompanying drawings, and are merely for the convenience of describing the present disclosure and simplifying the description, rather than indicating or implying that the referred device or element must have a specific orientation, be constructed and operated in a specific orientation, and therefore should not be construed as limiting the present disclosure.

In addition, when the terms “first” and “second” appear, these terms are used merely for descriptive purposes and are not to be construed as indicating or implying relative importance or implicitly specifying the number of the indicated technical features. Thus, a feature defined as “first” or “second” may explicitly or implicitly include at least one of that feature. In the description of the present disclosure, when the term “plurality” or “multiple” appears, the meaning of “plurality” is at least two (for example, two, three, etc.), unless otherwise expressly and specifically limited.

In the present disclosure, unless otherwise expressly provided and defined, when the terms “mounted,” “connected,” “linked,” “fixed,” etc. appear, these terms should be understood in a broad sense. For example, it may be a fixed connection, a detachable connection, or an integral connection; it may be a mechanical connection or an electrical connection; it may be a direct connection or an indirect connection through an intermediate medium; it may refer to internal communication between two elements or an interaction relationship between two elements, unless otherwise expressly limited. For a person of ordinary skill in the art, the specific meanings of the above terms in the present disclosure can be understood according to specific circumstances.

In the present disclosure, unless otherwise expressly provided and defined, when descriptions such as a first feature being “on” or “under” a second feature appear, this may mean that the first and second features are in direct contact, or that the first and second features are in indirect contact through an intermediate medium. Moreover, the first feature being “above,” “over,” or “on top of” the second feature may mean that the first feature is directly above or obliquely above the second feature, or may merely indicate that the horizontal height of the first feature is greater than that of the second feature. The first feature being “below,” “under,” or “beneath” the second feature may mean that the first feature is directly below or obliquely below the second feature, or may merely indicate that the horizontal height of the first feature is less than that of the second feature.

It should be noted that when an element is referred to as being “fixed to” or “disposed on” another element, it may be directly on the other element or an intervening element may also be present. When an element is considered to be “connected” to another element, it may be directly connected to the other element or an intervening element may be present at the same time. Where applicable, the terms “vertical,” “horizontal,” “upper,” “lower,” “left,” “right,” and similar expressions used in the present disclosure are for illustrative purposes only and do not indicate that they are the only possible embodiments.

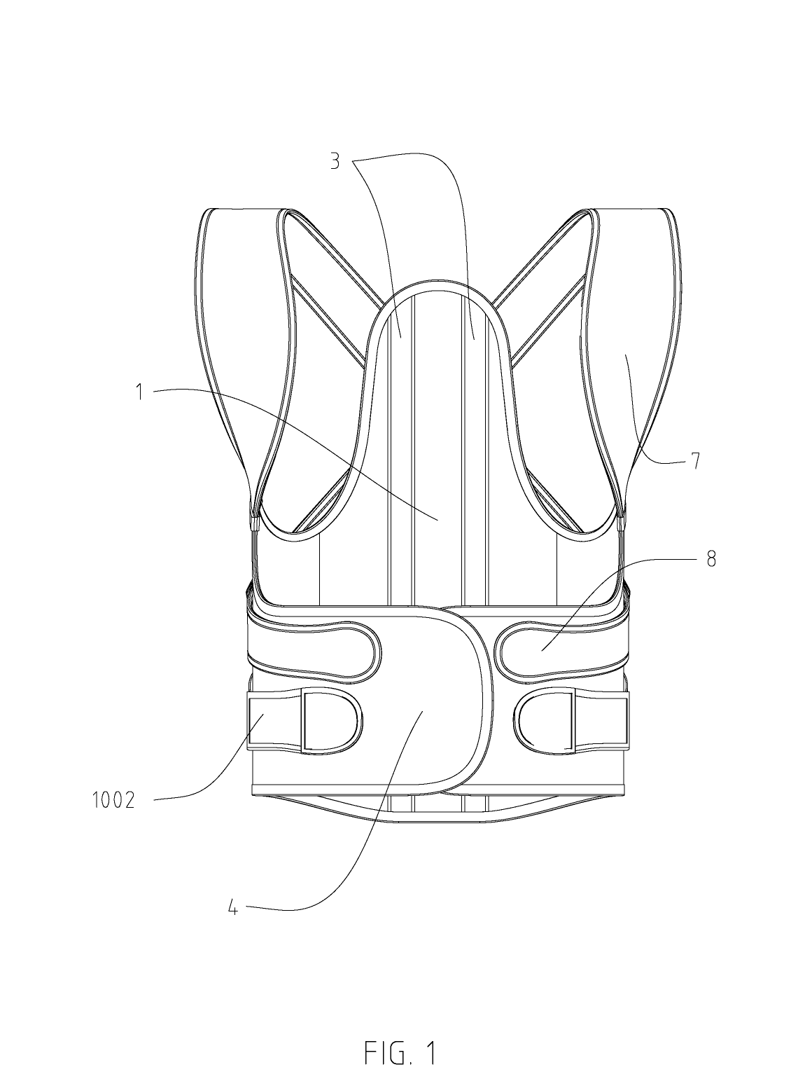

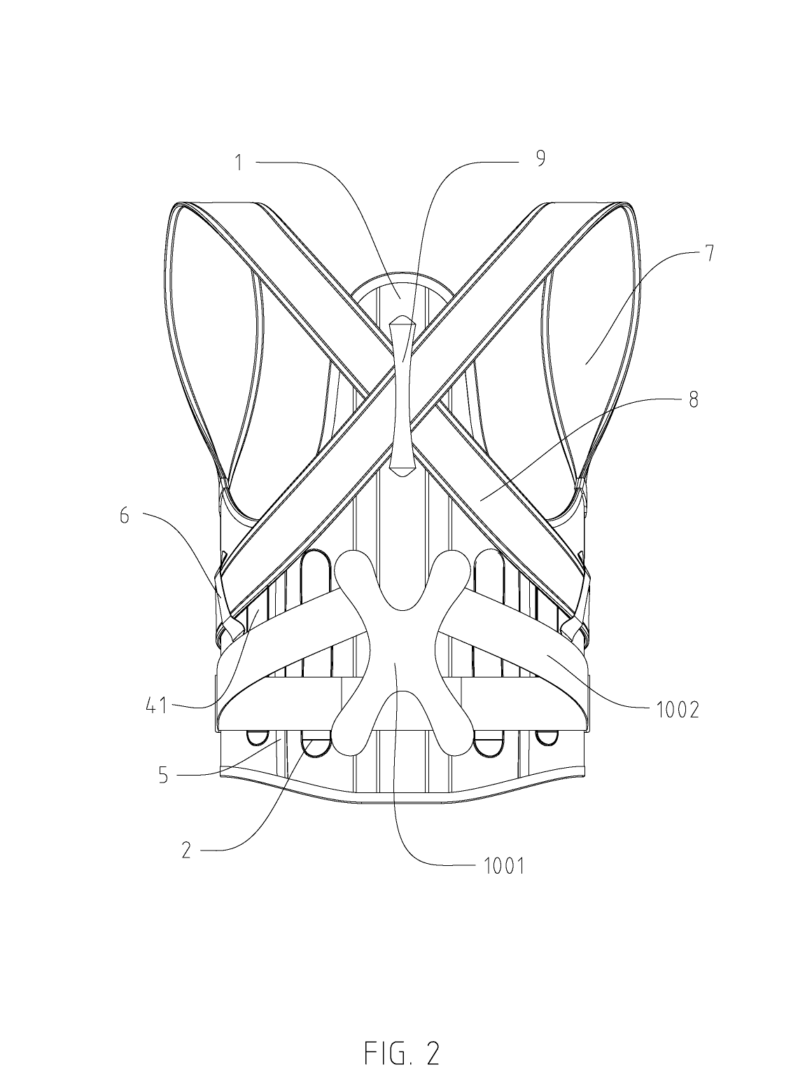

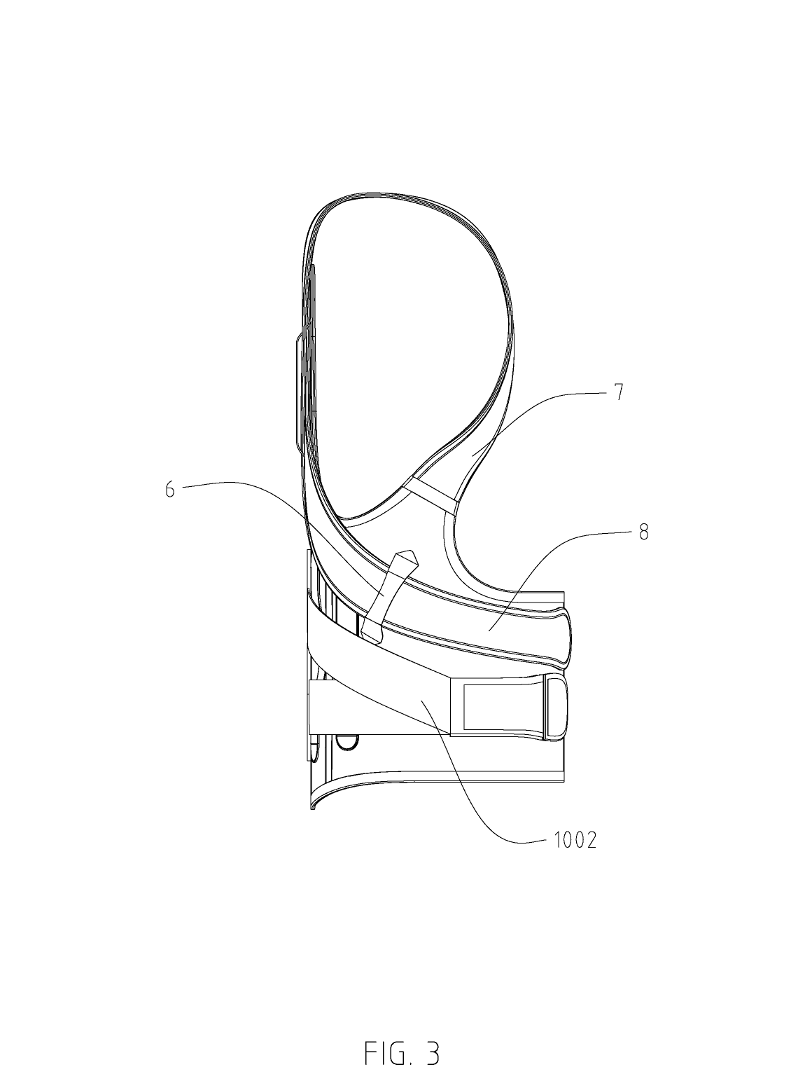

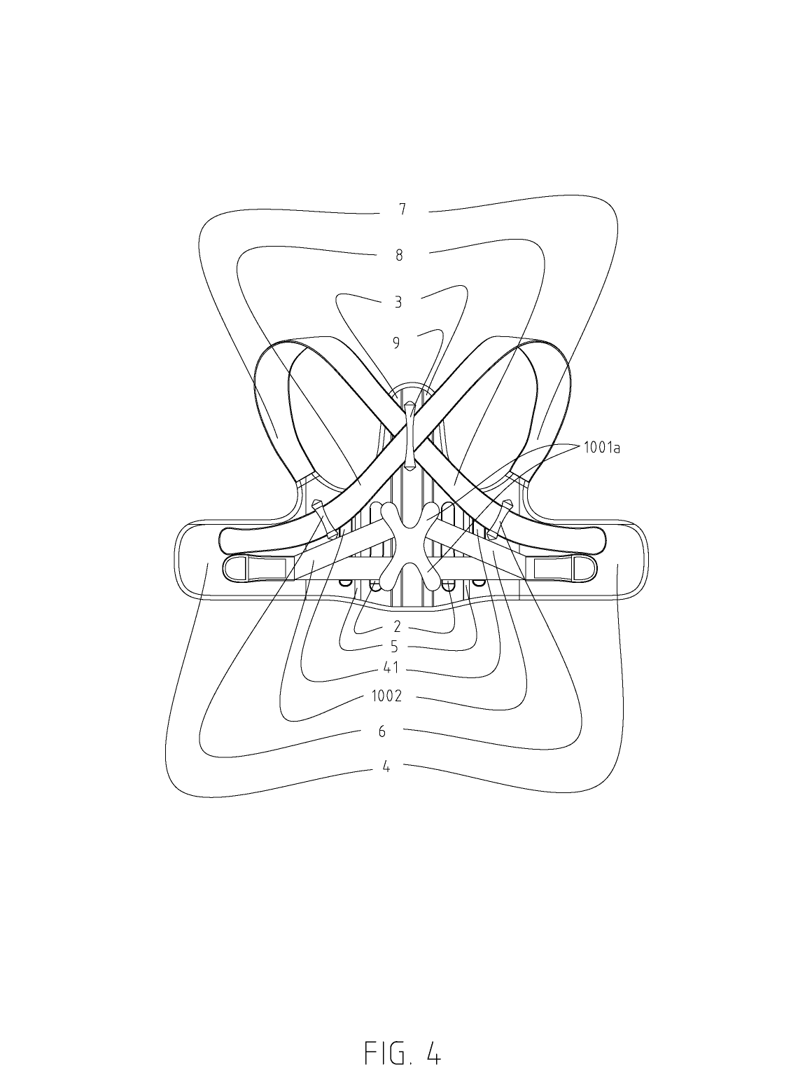

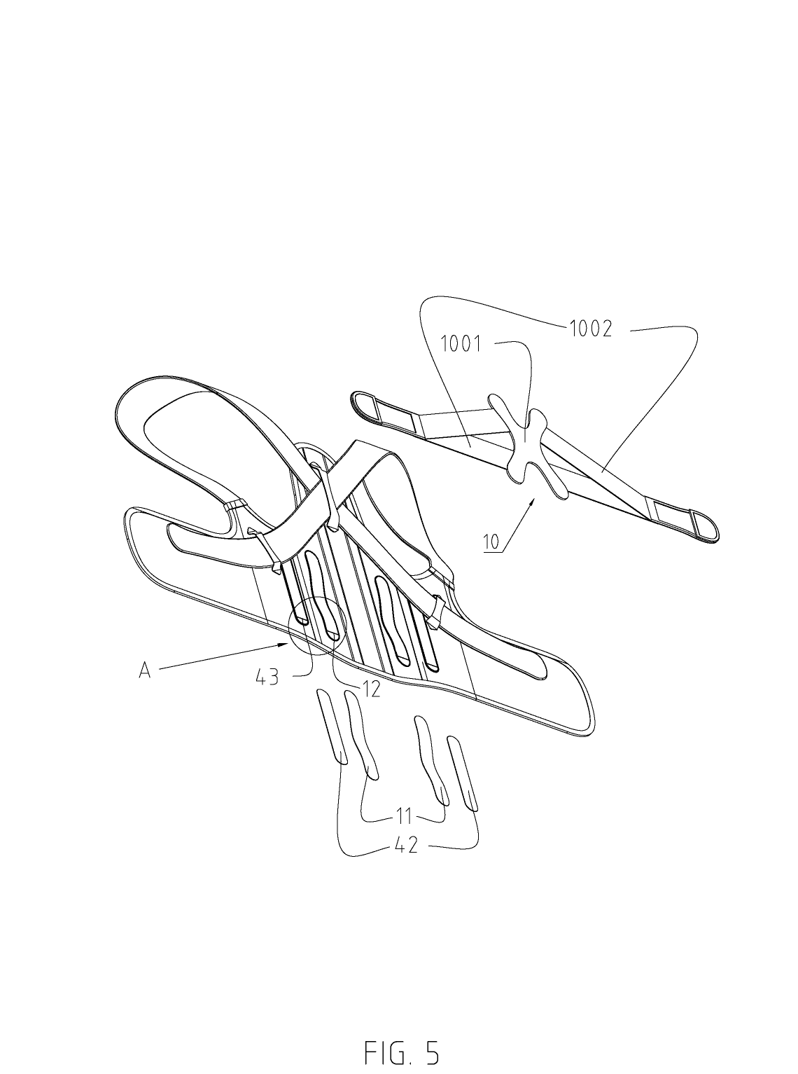

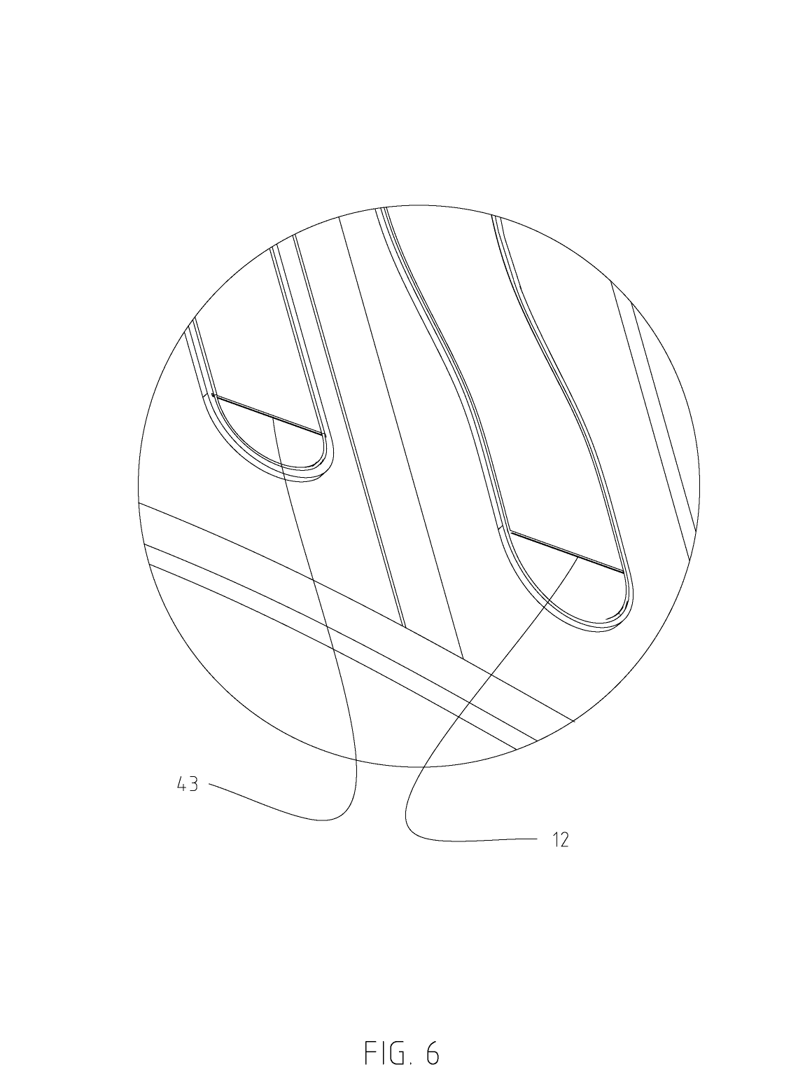

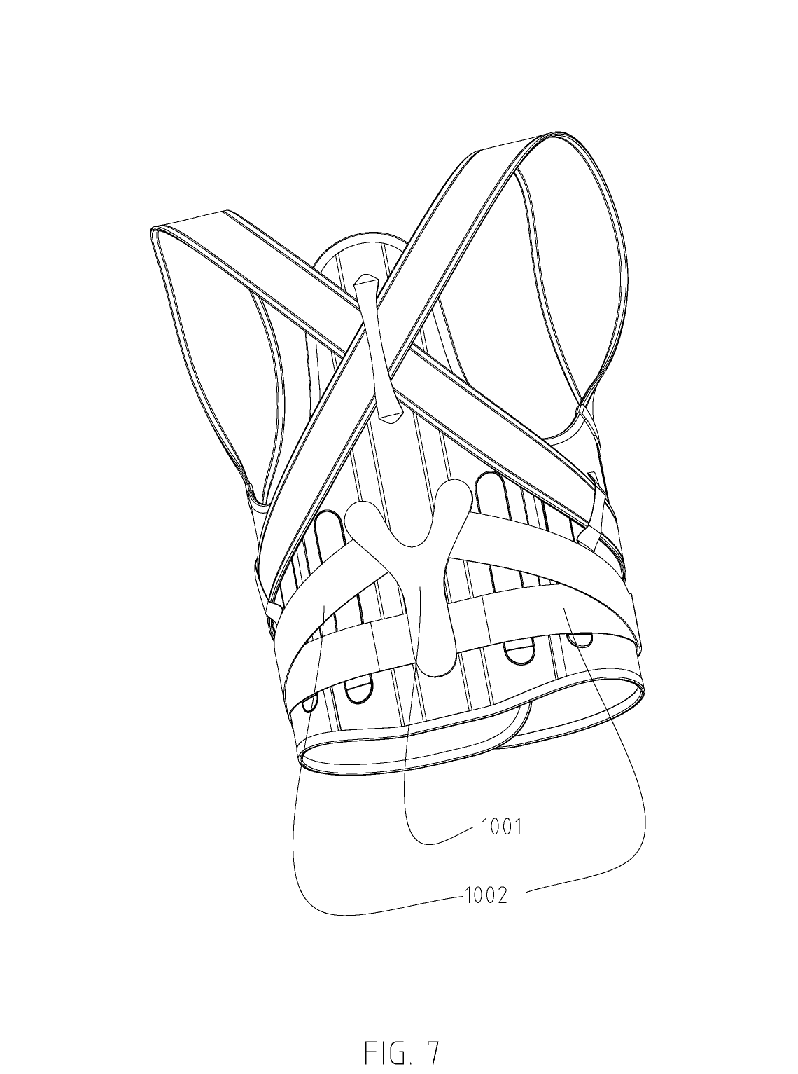

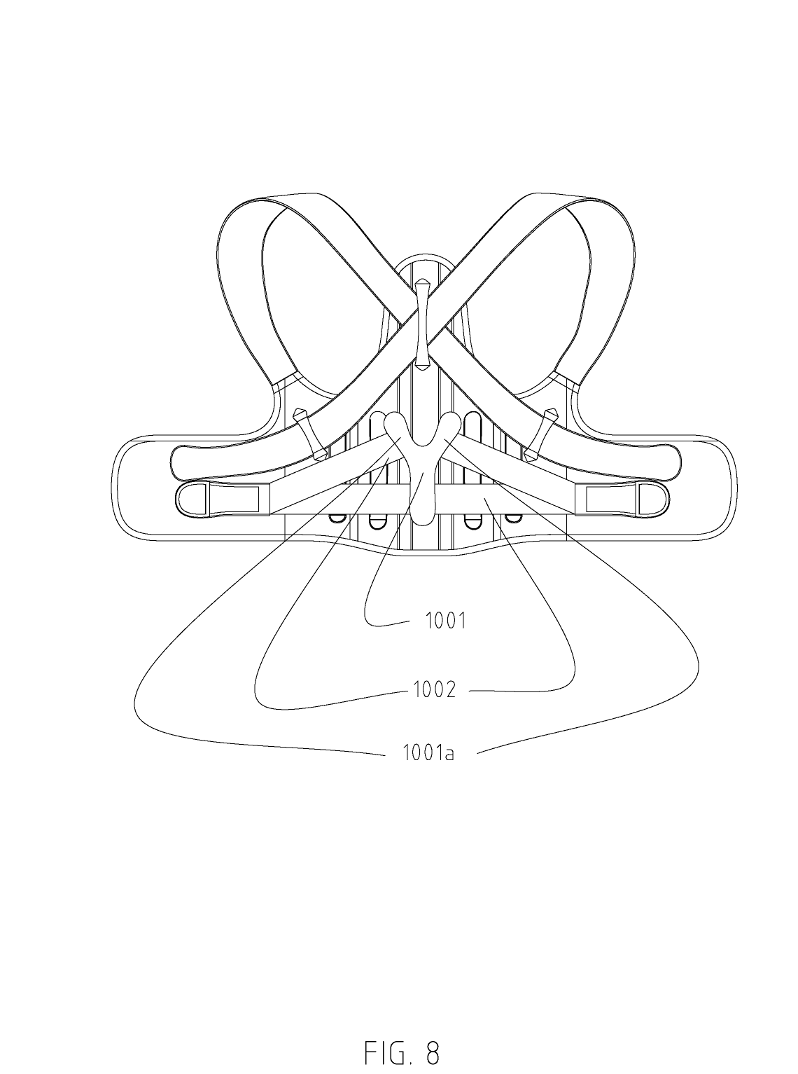

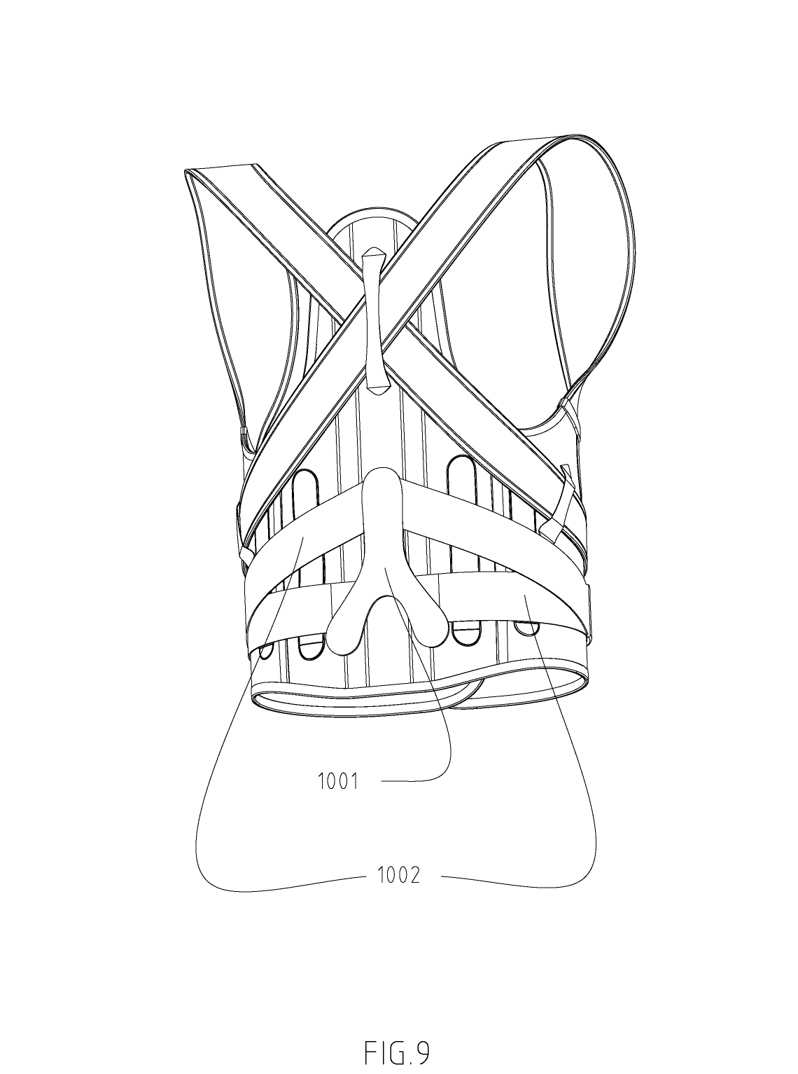

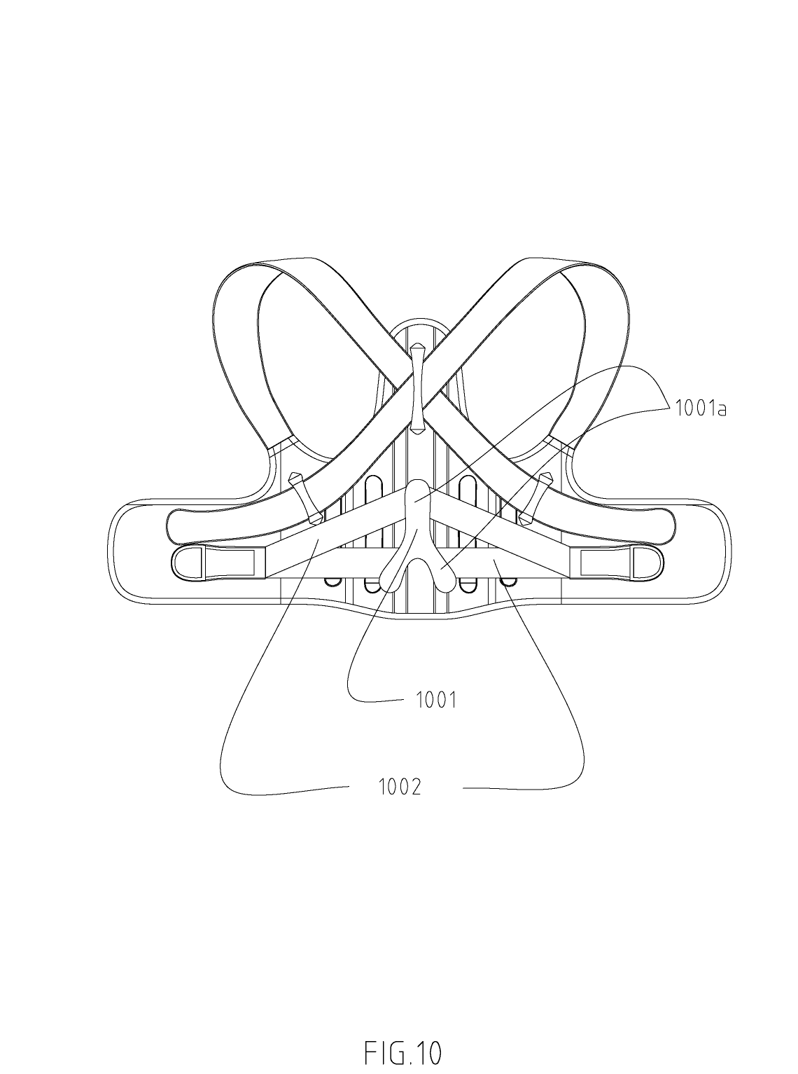

Referring to FIG. 1 to FIG. 10, in a first embodiment a posture correction device includes the following technical features.

A main orthopedic band 1 having at least two main support rods 3 embedded therein along a longitudinal direction of a spine, the at least two main support rods 3 are parallel arranged.

Two abdominal bands 4, symmetrically and adjustably connected to lower portions of both sides of the main orthopedic band 1, configured to encircle an abdomen of a user, each of the two abdominal bands 4 having an outer surface provided with a hook and loop fastener loop surface area.

Two shoulder straps 7, symmetrically and fixedly connected to upper portions of both sides of the main orthopedic band 1.

A back limiting portion 9, fixedly disposed on a side of the main orthopedic band 1 facing away from a body of the user.

Two elastic pulling straps 8, each of the two pulling straps 8 having a first end connected to a back portion of a corresponding one of the two shoulder straps 7. A second end of each of the two pulling straps sequentially slidably passes through the back limiting portion 9 and extends downward. The two pulling straps 8 cross in an X-shape at the back limiting portion 9. A distal end lower surface of each of the two pulling straps 8 is provided with a hook and loop fastener hook surface.

A lumbar pressure band 10, detachably connected to the main orthopedic band 1, the lumbar pressure band 10 comprising a support component 1001 and a plurality of elastic connecting straps 1002, wherein a proximal end of each of the plurality of elastic connecting straps 1002 is connected to the support component 1001, and a distal end thereof is detachably connected to a corresponding one of the two abdominal bands 4.

A restraining state of each of the two shoulder straps 7 on a shoulder of the user is adjusted by changing an attachment position of the hook and loop fastener hook surface at the distal end of each of the two pulling straps 8 on the hook and loop fastener loop surface area of a corresponding one of the two abdominal bands 4.

Through the arrangement of the above structure, in use, the user only needs to perform the core operation of changing the attachment position of the hook and loop fastener hook surface at the distal ends of the two pulling straps 8 on the hook and loop fastener loop surface area of the two abdominal bands 4, which can trigger a systematic mechanical adjustment process: the traction system constituted by the two elastic pulling straps 8 and the two shoulder straps 7 generates dynamic backward pulling force → the support system constituted by the main orthopedic band 1 and the embedded two main support rods 3 provides longitudinal stability → the detachable lumbar pressure band 10 provides forward pressurization and stabilization of the lumbar spine. The three act synergistically and jointly on the user’s shoulder, back, and waist regions.

This effectively addresses the pain points of traditional posture correction devices, such as fixed correction modes and single adjustment dimensions, and realizes a precise correction logic of position adjustment and force line optimization. In particular, the lumbar pressure band 10 is detachably connected to the main orthopedic band 1, which allows the lumbar support module to be replaced or adjusted independently of the main body. The user can flexibly choose according to their own lumbar curvature or the support needs of the day, achieving a leap from passively adapting to the product to the product actively adapting to the user.

By threading the two elastic pulling straps 8 through the back limiting portion 9 in an X-shaped crossing manner, the backward and downward pulling force on the shoulders is skillfully optimized and converted into an inward adduction force that symmetrically acts on the inner sides of both scapulae and converges toward the spine, which better conforms to the physiological mechanics of correcting rounded shoulders and kyphosis (hunchback). At the same time, the stepless adjustment mechanism achieved by changing the hook and loop attachment position enables continuous and fine control of the magnitude and point of application of the corrective force, avoiding the abrupt feeling of traditional segmented adjustments, and improving the smoothness and comfort of force transmission.

The main orthopedic band 1 has embedded therein parallel main support rods 3, which collectively constitute a reliable longitudinal rigid support structure. This configuration effectively restricts excessive kyphosis in the thoracolumbar spine segment and provides a stable force-bearing reference baseline for the entire correction system. The detachably connectable lumbar pressure band 10 employs a combined design including a support component 1001 and a plurality of elastic connecting straps 1002. This arrangement enables concentrated pressure applied to the lumbar vertebrae to be effectively distributed and transformed into a distributed surface load. Consequently, while providing the necessary stabilization, the design significantly reduces the risks of localized pressure concentration and associated discomfort.

The two shoulder straps 7 are fixedly connected to the main orthopedic band 1, the two abdominal bands 4 are adjustably wrapped around the torso, and the end positions of the two pulling straps 8 are steplessly adjustable. This structural cooperation ensures that the posture correction device can precisely conform to users of varying body types. In particular, the independent detachability of the lumbar pressure band allows the user to perform individualized setting or complete removal of lumbar support without affecting the shoulder and back correction function. This feature substantially enhances the product's adaptability and practicality across different usage scenarios, such as those requiring only shoulder-back posture reminding or those necessitating comprehensive lumbo-dorsal support. The overall adjustment mechanism is intuitive, requiring merely repositioning of the attachment locations, thereby markedly reducing the user's learning curve and operational complexity.

On this basis, the posture correction device further improves personalization and adaptability of correction. The user can not only finely control the traction force on the shoulder and back by altering the anchoring points of the pulling straps 8, but can also independently select or adjust the lumbar pressure band 10 to match varying requirements ranging from daily posture maintenance to targeted lumbar vertebral support. This modular and customizable design enables the posture correction device to dynamically adapt to different stages of the user's correction process, thereby facilitating consolidation of long-term correction outcomes and enhancing overall wearing experience. Through organic linkage among three subsystems—elastic traction, rigid support, and modular pressurization—the overall structure establishes a scientifically oriented, rationally distributed, and highly personalized three-dimensional correction solution.

In the present embodiment, the support component 1001 is provided with a plurality of connection sections 1001a. The proximal end of each of the elastic connecting straps 1002 are connected to one of the plurality of connection sections 1001a, and the total number of elastic connecting straps 1002 is greater than or equal to a number of connection sections 1001a. By virtue of the aforementioned connection relationship, the support component 1001 and its connection sections 1001a together form a flexibly expandable pressure distribution hub. This configuration allows a limited number of the connection sections 1001a to connect to and control a greater number of elastic connecting straps 1002, thereby efficiently decomposing the concentrated pressure exerted by the support component 1001 into component forces in multiple directions and angles. This not only improves the uniformity and dispersion of pressure transmission from the support component to the lumbar-abdominal region, avoiding excessive local pressure, but more importantly, provides a physical foundation for achieving individualized fine adjustment of pressure distribution.

In the present embodiment, the number of elastic connecting straps 1002 is an even number and they are arranged symmetrically with respect to the longitudinal centerline of the support component 1001. The number of elastic connecting straps 1002 is at least four, while the number of connection sections 1001a is at least three. Through the provision of symmetrical arrangement and at least four connecting straps1002, the lumbar pressure band 10 is capable of forming a completely mirrored and sufficiently dense distribution of force lines on the left and right sides of the user's lumbar vertebrae. Such symmetry ensures that the restraining forces applied to both sides of the spine remain constantly balanced, effectively preventing bodily compensation or discomfort that might otherwise result from pressure imbalance, thereby constituting a fundamental basis for mechanical stability. The lower limit of at least four straps ensures effective transmission of pressure from the center of the support component 1001 to the lateral and even anterior aspects of the lumbar-abdominal region, thereby forming a preliminary encircling pressure surface and avoiding mere point-like or linear pressure concentration solely at the central posterior waist. At the same time, setting the number of connection sections 1001a to at least three, in combination with at least four connecting straps, inevitably results in a multi-to-one configuration in which certain connection sections 1001a are connected to multiple straps. This design creates an efficient pressure conduction pathway from concentration to divergence: a single connection section1001a—particularly one located on the longitudinal axis of the support component—can serve as a force origin while simultaneously branching into multiple force lines in different directions (e.g., obliquely upward, obliquely downward, or horizontally). This greatly optimizes force dispersion efficiency and enables the support component 1001, in a relatively compact and ergonomically conforming shape (such as an X-shape, Y-shape, or inverted Y-shape), to achieve uniform and three-dimensional coverage and support over a large area of the waist region through only a limited number of anchor points.

In the present embodiment, the main orthopedic band 1 is provided with at least one two auxiliary support mechanisms symmetrically disposed on both sides of the main support rods 3. The auxiliary support mechanisms are auxiliary support buckles 2, each of which removably accommodates an auxiliary supporting rod 11. The auxiliary supporting rod 11 includes an arcuate segment adapted to the physiological curvature of the lumbar vertebrae. With the above structural arrangement, the auxiliary support system provides supplementation and refined adjustment to the primary support system. Firstly, the symmetrically arranged auxiliary support buckles 2 and the removable auxiliary supporting rods 11 together construct, within the main orthopedic band 1, a stable support framework consisting of one primary and two auxiliary elements. The main support rods 3 bear the core longitudinal load of the spine, while the auxiliary supporting rods 11 on both sides supply critical lateral stabilizing forces, jointly resisting lateral bending and torsion of the trunk and significantly enhancing the overall structural resistance to deformation and stability. Secondly, the removable design of the auxiliary supporting rods 11 imparts personalized fitting capability to the product. Depending on the user's actual requirements for lateral support strength (for example, stronger support during the early stage of correction or targeted reinforcement at specific lumbar segments). The user can flexibly choose to insert or remove the auxiliary supporting rods 11, or even replace rods of different stiffness or curvature.

In the present embodiment, each of the auxiliary supporting rods 11 is removably inserted into or removed from each of the auxiliary support buckles 2 through a first opening 12 provided on each of the auxiliary support buckle 2. This enables a standardized product to precisely match the differentiated needs of different users and different correction stages, thereby overcoming the drawback of conventional integrated posture correction devices in which support strength is either excessively strong or excessively weak and incapable of dynamic adjustment. Furthermore, the arcuate segment of each of the auxiliary supporting rods 11, which is adapted to the physiological curvature of the lumbar vertebrae, allows the rigid support element to actively conform to the natural lordotic curve of the human waist. During wearing, this arcuate segment can establish surface contact rather than point contact or linear contact with the lumbar vertebral region of the user. Thereby distributing the supporting force uniformly over a larger body surface area and greatly avoiding localized pressure or gaps between the support and body contours that might occur with straight-bar-shaped support components. This not only improves wearing comfort in the lumbar region, but more importantly, ensures that the direction of the applied supporting force remains perpendicular to and compliant with the physiological curvature at all times. Thereby realizing the scientific principle of “support equals correction” and more safely and effectively assisting in maintaining or restoring the normal physiological lordos is of the lumbar vertebrae.

In this embodiment, each of the two abdominal bands 4 is provided with one rib limiting strap 6. Each of the two pulling straps 8, after passing through the back limiting portion 9, further passes through the rib limiting strap 6 on the same side. Through the above arrangement, the rib limiting strap 6 serves as the second key guiding point along the sliding path of a corresponding the two pulling straps 8, thereby enabling a secondary optimization and precise control over the direction of the corrective force. After emerging from the back limiting portion 9, the extension direction of each of the two pulling straps 8 is further guided and fixed by a corresponding rib limiting strap 6. At the same time, the rib limiting strap 6 functions as a movable anchor point for the corresponding two pulling straps 8, thereby enhancing the overall stability of the traction system. It prevents arbitrary swinging or twisting of the excessively long segment of each of the two pulling straps 8 between the back and the abdomen, ensures continuous and stable transmission of the corrective force, avoids deviation of the force line due to body movements, and thereby guarantees the persistence and consistency of the corrective effect.

In this embodiment, the hook and loop fastener loop surface area of each of the abdominal bands 4 covers a major portion of its length, such that the hook surface at the distal end of each of the two pulling straps 8 can be continuously positionally adjusted along the longitudinal direction of a corresponding one of the two abdominal band 4. The continuous and integral loop surface region allows the end of the two pulling straps 8 to be precisely positioned in a manner analogous to a sliding switch, thereby enabling the user to exercise extremely fine linear control over the tensile force applied to the shoulder and back regions. This adjustment method eliminates the stepped, discrete feeling of conventional positioning, allowing the user to accurately set a personalized balance point between effective correction and imperceptible comfort, which significantly improves long-term wearing tolerance and user compliance.

The coverage of the major portion means that the user can freely select the attachment point within a wide area extending from the middle-rear to the front portion of each of the two abdominal bands 4. This arrangement resolves the problems inherent in conventional designs where the attachment area is narrow or consists of discrete points, which result in limited adjustment range and poor adaptability to diverse body types. The wide-range continuous adjustment track enables users of greatly varying physiques—from petite to robust—to locate the optimal anchoring point that best matches their individual anatomical dimensions and moment arm requirements.

In this embodiment, both the rib limiting strap 6 and the back limiting portion 9 are made of microfiber leather. The smooth and wear-resistant surface characteristics of the microfiber leather provide favorable conditions for the sliding of the two pulling straps 8 therethrough, significantly reducing the frictional resistance between the two pulling straps 8 and the limiting strap 6 as well as the back limiting portion 9 during passage and adjustment. This makes the operation of adjusting the attachment position markedly smoother and more labor-saving for the user. Meanwhile, the excellent wear resistance ensures that long-term and frequent sliding friction does not easily cause surface wear, burr formation, or other damage to the components, thereby maintaining long-lasting smoothness of the force transmission path and superior durability of the product.

In this embodiment, the lumbar pressure band 10 is detachably connected to the main orthopedic band 1 by means of a hook and loop fastener assembly. The outer surface of the main orthopedic band 1 is provided with a loop surface of the hook and loop fastener, while the surface of the support component 1001 is provided with a hook surface of the hook and loop fastener.

Through the hook and loop fastener assembly described above, a physical and functional connection between the lumbar pressure band 10 module and the main system is achieved that is both secure and reliable as well as extremely convenient. Firstly, the user only needs to align the hook surface on the back of the support component 1001 with the corresponding loop surface in the lumbar region of the main orthopedic band (1) and press it into place, thereby completing the connection. This connection method requires no tools or complicated operations, can be accomplished within seconds, and provides sufficient shear resistance through large-area surface contact, ensuring that the lumbar pressure band 10 will not unintentionally shift or detach during activities and thereby maintaining the integrity and functional stability of the orthopedic system. Secondly, it endows the user with a high degree of personalized configuration freedom. Because the connection is based on the face-to-face bonding principle of hook and loop fastener, the user can finely adjust the vertical (superior-inferior) attachment height of the lumbar pressure band 10 so as to precisely match the most prominent point of the physiological lumbar lordosis or the spinal segment most in need of support. This vertical adjustability, combined with the horizontal tightness adjustment of the lumbar pressure band itself achieved through the elastic connecting straps 1002, enables three-dimensional personalized adaptation of lumbar support, greatly enhancing the precision and comfort of the correction.

In this embodiment, a position of each of the two abdominal bands 4 proximate to the main orthopedic band 1 is provided with an abdominal band support buckle 41. The abdominal band support buckle 41 removably accommodates an abdominal band support rod (42). At least one shaping support rod 5 is embedded at a junction between the two abdominal bands 4 and the main orthopedic band.

Through the above arrangement, the detachable abdominal band support rod 42 and the at least one shaping support rod 5 together form a synergistic “point-line” reinforcement structure. The at least one shaping support rod 5 is longitudinally arranged along the connection seam between the two abdominal bands and the main base band, effectively reinforcing this critical mechanical interface line for force transmission, preventing inappropriate bending or stress concentration at the connection during circumferential tightening around the waist or when subjected to tension from the two pulling straps. Thereby ensuring smooth and efficient transmission of force from the lateral trunk to the main back support system. The detachable design of the abdominal band support rod 42 realized through the second opening 43 once again embodies the overall modular and customizable philosophy of the posture correction device. The user can flexibly adjust the support stiffness characteristics of this region according to their specific needs for lateral waist-abdomen support rigidity (for example, inserting the abdominal band support rod 40 when strong abdominal drawing-in support is required or when there is lumbar injury necessitating additional reinforcement, or removing it during routine mild use to maximize flexibility and softness). This design transforms the two abdominal bands from a component with fixed functionality into a dynamically adaptable support interface that changes according to user requirements.

In this embodiment, the shape of the support component 1001 is X-shaped, Y-shaped, or inverted Y-shaped, and the material of the support component 1001 is leather. By defining the shape of the support component 1001 as one of the aforementioned specific configurations, an optimal geometric expression is substantially imparted to its mechanical topology. The common feature of the X-shaped, Y-shaped, and inverted Y-shaped designs is a concentrated central region with multiple outwardly radiating “arms.” This morphology perfectly aligns with the design concept of “number of connection straps ≥ number of connection sections”. The central region naturally serves as the primary force-origin point of the connection sections 1001a, facilitating efficient “many-to-one” force convergence. While the outwardly radiating “arms” provide clear and scientifically rational extension guidance for each elastic connecting strap 1002, ensuring that the connection straps can be distributed at appropriate angles, and thereby uniformly transmit the supporting force to different regions of the waist and abdomen. In particular, the Y-shaped or inverted Y-shaped configuration better conforms to the physiological contour of the lumbar vertebral region, wherein the central main trunk can be arranged along the spinal column direction, and the upper bifurcations or lower extensions can more effectively correspond to the upper lumbar segments or sacral region, thereby achieving form-follows-function precise support.

It should be noted that the distinction among the first embodiment, the second embodiment, and the third embodiment of the present disclosure lies in the different shapes of the support component 1001, which are respectively X-shaped, Y-shaped, and inverted Y-shaped.

In this embodiment, the two pulling straps 8 are made of elastic webbing, while the main body of the main orthopedic band 1, the two abdominal bands 4, and the two shoulder straps 7 are made of flexible textile materials. The two pulling straps (8) are fabricated from elastic webbing ensures the provision of dynamic, compliant, and sustainably maintained shoulder traction force, with its elastic property serving as the foundation for stepless adjustment and conformity during movement. At the same time, the main body of the main orthopedic band (1), the two abdominal bands (4), and the two shoulder straps (7) are made of flexible textile materials imparts excellent skin conformity, breathability, and durability to the product. The overall material system establishes a synergistic foundation of elastic dynamic traction combined with flexible-rigid composite support, which, while ensuring effective mechanical correction, maximally guarantees wearing comfort, natural range of motion, and long-term practical usability.

The foregoing descriptions present one or more embodiments in conjunction with specific content and are not intended to limit the specific implementations of the present disclosure to these illustrations. Any methods, structures, or the like that are approximate or identical to those of the present disclosure, or any technical deductions, substitutions, or modifications made based on the inventive concept of the present disclosure, shall be deemed to fall within the protection scope of the present disclosure.

What is claimed is:

1. A posture correction device, comprising:
a main orthopedic band having at least two main support rods embedded therein along a longitudinal direction of a spine, the at least two main support rods being parallel arranged to each other;
two abdominal bands symmetrically and adjustably connected to lower portions of both sides of the main orthopedic band, the two abdominal bands being configured to encircle an abdomen of a user, each of the two abdominal bands having an outer surface provided with a hook and loop fastener loop surface area;
two shoulder straps symmetrically and fixedly connected to upper portions of both sides of the main orthopedic band;
a back limiting portion fixedly disposed on a side of the main orthopedic band facing away from a body of the user;
two pulling straps, each of the two pulling straps having a first end connected to a back portion of a corresponding one of the two shoulder straps, and a second end of each of the two pulling straps sequentially slidably passes through the back limiting portion and extends downward, wherein the two pulling straps cross each other in an X-shape over the back limiting portion; and a distal end lower surface of each of the two pulling straps being provided with a hook and loop fastener hook surface;
a lumbar pressure band detachably connected to the main orthopedic band, the lumbar pressure band comprising a support component and a plurality of elastic connecting straps, wherein a proximal end of each of the plurality of elastic connecting straps is connected to the support component, and a distal end thereof is detachably connected to a corresponding one of the two abdominal bands;
wherein a restraining state of each of the two shoulder straps on a shoulder of the user is adjusted by changing an attachment position of the hook and loop fastener hook surface at the distal end of each of the two pulling straps relative to the hook and loop fastener loop surface area of a corresponding one of the two abdominal bands.

2. The posture correction device according to claim 1, wherein the support component is provided with a plurality of connection sections; the proximal end of each of the plurality of elastic connecting straps is connected to a respective one of the connection sections, and a number of the elastic connecting straps is greater than or equal to a number of the connection sections.

3. The posture correction device according to claim 2, wherein the number of the elastic connecting straps is an even number, and the elastic connecting straps are symmetrically arranged along a longitudinal centerline of the support component.

4. The posture correction device according to claim 2, wherein the number of the elastic connecting straps is at least four.

5. The posture correction device according to claim 4, wherein the number of the connection sections is at least three.

6. The posture correction device according to claim 1, wherein the main orthopedic band is provided with at least two auxiliary support mechanisms, symmetrically disposed on both sides of the main support rods.

7. The posture correction device according to claim 6, wherein the auxiliary support mechanisms are auxiliary support buckles, and each of the auxiliary support buckles removably accommodates an auxiliary supporting rod.

8. The posture correction device according to claim 7, wherein each of the auxiliary support rods comprises an arcuate segment adapted to a physiological curvature of a lumbar spine of the user.

9. The posture correction device according to claim 1, wherein each of the two abdominal bands is provided with a rib limiting strap, and after passing through the back limiting portion, each of the two pulling straps further passes through the rib limiting strap positioned on the same side.

10. The posture correction device according to claim 1, wherein the hook and loop fastener loop surface area of each of the two abdominal bands covers a major portion of its length, thereby facilitating continuous positional adjustment of the hook and loop fastener hook surface along a length direction of a corresponding one of the two abdominal bands.

11. The posture correction device according to claim 9, wherein a material of the rib limiting strap and the back limiting portion is microfiber leather.

12. The posture correction device according to claim 1, wherein the lumbar pressure band is detachably connected to the main orthopedic band by a hook and loop fastener assembly.

13. The posture correction device according to claim 12, wherein an outer surface of the main orthopedic band is provided with a hook and loop fastener loop surface, and a surface of the support component is provided with a hook and loop fastener hook surface.

14. The posture correction device according to claim 1, wherein an abdominal band support buckle is provided at each of the two abdominal bands close to the main orthopedic band.

15. The posture correction device according to claim 14, wherein an abdominal band support rod is detachably disposed in the abdominal band support buckle.

16. The posture correction device according to claim 14, wherein at least one shaping support rod is embedded at a junction between the two abdominal bands and the main orthopedic band.

17. The posture correction device according to claim 1, wherein a shape of the support component is X-shaped, Y-shaped, or inverted Y-shaped.

18. The posture correction device according to claim 1, wherein a material of the support component is leather.

19. The posture correction device according to claim 1, wherein the pulling strap is made of elastic webbing.

20. The posture correction device according to claim 1, wherein main bodies of the main orthopedic band, the two abdominal bands, and the two shoulder straps are made of flexible textile material.
